(19) United States
(12) Patent Application Publication
(10) Pub. No.: US 2023/0241170 A1
(43) Pub. Date: Aug. 3, 2023

(54) EPIDERMAL GROWTH FACTOR-LIKE 7 PEPTIDE AND USES THEREOF

(71) Applicants: Ohio State Innovation Foundation, Columbus, OH (US); RSEM, LIMITED PARTNERSHIP, Quebec (CA)

(72) Inventors: Adrienne DORRANCE, Columbus, OH (US); Martin GUIMOND, Columbus, OH (US)

(21) Appl. No.: 18/004,949

(22) PCT Filed: Jul. 9, 2021
(86) PCT No.: PCT/US2021/041122

§ 371 Date: Jan. 10, 2023

Related U.S. Application Data
(60) Provisional application No. 63/050,210, filed on Jul. 10, 2020.

(51) Int. Cl. A61K 38/18; A61P 37/06; A61K 31/519; A61K 38/13; A61K 31/436; A61K 31/5377; A61K 31/573; A61K 31/675; A61K 39/395

(52) U.S. Cl. CPC A61K 38/1808; A61P 37/06; A61K 31/519; A61K 38/13; A61K 31/436; A61K 31/5377; A61K 31/573; A61K 31/675; A61K 39/3955; A61K 39/39516

(57) ABSTRACT

The present disclosure relates to a recombinant endothelial growth factor like 7 (EGFL7) peptide and uses thereof for treating graft-versus-host disease (GVHD).

### CROSS REFERENCE TO RELATED APPLICATIONS

This application claims the benefit of U.S. Provisional Patent Application Ser. No. 63/050,210 filed Jul. 10, 2020, the disclosure of which is expressly incorporated herein by reference.

### FIELD

The present disclosure relates to epidermal growth factor-like 7 (EGFL-7) peptides and uses thereof.

### BACKGROUND

Graft-versus-host disease (GVHD) is a frequent and lethal complication of allogeneic hematopoietic stem cell transplantation (HSCT) in which donor T cells destroy HLA mismatched host tissues by secreting inflammatory cytokines (TNF-α and IFN-γ) and/or inducing direct cytotoxic cellular responses. Despite recent advances, GVHD still remains a major clinical problem, underscoring the need to elucidate further its mechanisms to then develop novel therapeutic strategies. What is needed are compositions and methods for treating GVHD. The compositions and methods disclosed herein address these and other needs.

### SUMMARY

In some aspects, disclosed herein is a method of treating graft-versus-host disease (GVHD) in a subject, comprising administering to the subject a therapeutically effective amount of a recombinant endothelial growth factor like 7 (EGFL7) peptide or fragment thereof.

In some embodiments, the recombinant EGFL7 peptide comprises a polypeptide sequence at least 80% identical to SEQ ID NO: 1. In some embodiments, the recombinant EGFL7 peptide comprises the polypeptide sequence of SEQ ID NO: 1. In some embodiments, the recombinant EGFL7 peptide comprises a polypeptide sequence comprising at least 10 consecutive amino acids of SEQ ID NO: 1.

In some embodiments, the recombinant EGFL7 peptide is administered daily. In some embodiments, the recombinant EGFL7 peptide is administered prior to and/or after bone marrow transplantation. In some embodiments, the recombinant EGFL7 peptide is administered intravenously.

In some embodiments, the recombinant EGFL7 mitigates a symptom of GVHD, wherein the symptom comprises skin rashes, abdominal cramps, nausea, or liver injury. In some embodiments, the recombinant EGFL7 peptide induces T cell exhaustion and/or T cell tolerance in the subject. In some embodiments, the recombinant EGFL7 peptide decreases a level of an inflammatory cytokine and/or an adhesion molecule in the subject. In some embodiments, the inflammatory cytokine comprises TNF-α or IFN-γ. In some embodiments, the adhesion molecule comprises ICAM-1 or VCAM-1.

In some embodiments, the recombinant EGFL7 disclosed herein is formulated in a pharmaceutically acceptable carrier.

In some embodiments, the method of any preceding aspect further comprises administering to the subject a therapeutically effective amount of an additional agent for treating GVHD. In some embodiments, the additional agent is selected from the group consisting of methotrexate, cyclosporine, tacrolimus, mycophenolate mofetil, sirolimus, corticosteroid, antithymocyte globulin, alemtuzumab, and cyclophosphamide

### BRIEF DESCRIPTION OF THE DRAWINGS

The accompanying figures, which are incorporated in and constitute a part of this specification, illustrate several aspects described below.

FIGS. 1A-1J shows that recombinant EGFL7 therapy diminishes GVHD severity. (FIG. 1A) Schematic representation of the experimental design in which B6D2F1 mice were transplanted with bone marrow (BM) from C57BL/6 mice and alloreactive T cells from B6SJL mice. At day +21 post-allo-BMT, transplanted mice received daily injection of rEGFL7 or PBS. (FIG. 1B) Weights of animals that had undergone transplantation were measured daily and averaged for the group (circles: syngeneic T cells (no GVHD): Triangles: allogeneic treated with PBS; squares: allogeneic treated with rEGFL7. Measures were compared as a percentage change from day 0 and were pooled from 3 experiments with 6 to 13 mice per group. (FIG. 1C) Survival curve of transplanted mice (Grey line: syngeneic control (SYN), black line allogeneic treated with rEGFL7 and thin dotted line allogeneic treated with PBS). Data was pooled from 3 experiments with 6 to 13 mice per group. Mice were euthanized between day 28-31 post-HSCT. (FIG. 1D) Visual examination of the colon. (FIG. 1E) Histogram representative of the length of the colon of SYN, allogeneic treated with rEGFL7 and allogeneic treated with PBS. Data were pooled from 3 experiments with 4 to 8 mice per group (FIG. 1F) Histopathology of the gut and liver after allo-HSCT. (FIG. 1G) Histopathology score of the gut and (FIG. 1H) liver of rEGFL7 and PBS treated mice. Results show mean±SEM. Immunofluorescence analysis of the intestine from vehicle (PBS) or rEGFL7 treated mice transplanted with TCDBM+allogenic splenocytes were stained for immunofluorescence. (FIG. 1I) Cells were stained with CD31 and Ki67 antibodies. Nuclei were counterstained with DAPI. Number of CD31+ and Ki67+ cells were counted in 2 random fields per mouse and percentage of Ki67+CD31+ cells was calculated. N=3-4 mice per group, **P<0.01. (FIG. 1J) Number of CD3+CD4+ and CD3+CD8+ T cells were counted in 3 random fields per mouse. N=4 mice per group, **P<0.0.

FIGS. 2A-2I shows the effect of recombinant EGFL7 on immune cells. (FIG. 2A) Absolute counts of TCR+, CD4+ and CD8+ lymphocytes in the spleen of SYN, GVHD+rEGF-L7 and GVHD+PBS treated mice. Data were pooled from 3 experiments with 4 to 7 mice per group (FIG. 2B) Absolute counts of CD11c+DCs (left panel), B220+ B cells in the spleen (middle panel), thymocytes in the thymus (right panel). Data were pooled from 3 experiments with 4 to 8 mice per group. Results show mean f SEM. P values were determined by a Kruskal-Wallis test followed by a post-hoc Dunn's test (*P≤0.05; **P≤0.01). (FIG. 2C) Dot plot analysis of DCs, based on the expression of CD11c and CD11b antigens, in the spleen of SYN control, GVHD+rEGFL7 and GVHD+PBS mice. (FIG. 2D) Dot plot analysis B cells, based on the expression of CD19 and B220 antigens, in the spleen of SYN control, GVHD+rEGFL7 and GVHD+PBS mice. (FIG. 2E) Dot plot analysis of thymocytes, based on the expression of CD4 and CD8 antigens, in the thymus of SYN control, GVHD+rEGFL7 and GVHD+PBS mice. These data are representative of 3 or more experiments with 6 to 8 mice per group. Histogram showed mean+/−error type. Transplant for GVL was performed as described in methods. (FIG. 2F-FIG. 2G) Whole-body bioluminescent signal intensity of recipient mice (n=4-5 per cohort). Mice were imaged on indicated days. Average radiance expressed as mean±SEM. One representative transplant experiment of two is shown. (FIG. 2H) Percentage GFP positivity representing P815 leukemic cell infiltration in the spleen. Each dot represents a single mouse. (FIG. 2I) Representative flow cytometric contour plots. ***p<0.001.

FIGS. 3A-3G show results for B6D2F1 mice were transplanted with bone marrow (BM) from C57Bl/6 mice and alloreactive T cells from B6SJL mice. (FIG. 3A) Schematic representation of the experimental design in which B6D2F1 mice were transplanted with bone marrow (BM) from C57BL/6 mice and alloreactive T cells from B6SJL mice. At day +21 post-allo-BMT, transplanted mice received daily injection of rEGFL7 or PBS. (FIG. 3B) Visual examination of the colon. (FIG. 3C) Histogram representative of the length of the colon of SYN, allogeneic treated with rEGFL7 and allogeneic treated with PBS, *P≤0.05. Data were pooled from 3 experiments with 4 to 8 mice per group. Results show mean t SEM. P values were determined by a Kruskal-Wallis test followed by a post-hoc Dunn's test (*P≤0.05) (FIG. 3D) Histopathology of the liver after allo-HSCT. (FIG. 3E) Histopathology score of the liver of rEGFL7 and PBS-treated mice, *P≤0.05. (FIG. 3F) Results show mean t SEM. Number of CD31⁺ and Ki67⁺ cells were counted in 2 random fields per mouse and percentage of Ki67⁺CD31⁺ cells was calculated. N=3-4 mice per group, **P≤0.01. (FIG. 3G) Number of CD3⁺CD4⁺ and CD3⁺CD8⁺ T cells were counted in 3 random fields per mouse. N=4 mice per group, **P≤0.01.

FIGS. 4A-4C show lethally irradiated BALB/c recipients received T cell depleted bone marrow cells (TCD-BM, 10×10⁶ cells) along with B6 T cells (1×10⁶). Two weeks after transplant, recipients were divided into 3 cohorts—no treatment, PBS vehicle or rEGFL7 administered intraperitoneally 5 times a week for 5 weeks. (FIG. 4A) Transplant schema. (FIG. 4B) Survival curve, p<0.05. Log-rank test was used to compare survival. Data pooled from two independent experiments with 5-9 mice per group. (FIG. 4C) Acute GVHD Clinical scores. One representative transplant experiment of two is shown. *p<0.05.

FIGS. 5A-5E show immune reconstitution in GVHD mice. (FIG. 5A-FIG. 5B) Absolute counts of CD11c⁺ DCs (left panel), B220⁺ B cells in the spleen (middle panel), Data were pooled from 3 experiments with 4 to 8 mice per group. Results show mean f SEM. P values were determined by a Kruskal-Wallis test followed by a post-hoc Dunn's test (*P≤0.05; **P≤0.01). (FIG. 5C-FIG. 5E) Absolute counts of BM cells, CD11C DCs and CD19⁺220⁺ B cells in the BM of SYN, GVHD+EGFL7 and GVHD+PBS treated mice. These data are representative of 3 or more experiments with 6-10 mice per group.

### DETAILED DESCRIPTION

Reference will now be made in detail to the embodiments of the invention, examples of which are illustrated in the drawings and the examples. This invention may, however, be embodied in many different forms and should not be construed as limited to the embodiments set forth herein.

Unless defined otherwise, all technical and scientific terms used herein have the same meaning as commonly understood to one of ordinary skill in the art to which this disclosure belongs.

### Terminology

Terms used throughout this application are to be construed with ordinary and typical meaning to those of ordinary skill in the art. However, Applicant desires that the following terms be given the particular definition as defined below.

As used herein, the article “a,” “an,” and “the” means “at least one,” unless the context in which the article is used clearly indicates otherwise.

The terms “about” and “approximately” are defined as being “close to” as understood by one of ordinary skill in the art. In one non-limiting embodiment, the terms are defined to be within 10%. In another non-limiting embodiment, the terms are defined to be within 5%. In still another non-limiting embodiment, the terms are defined to be within 1%.

“Administration” to a subject includes any route of introducing or delivering to a subject an agent. Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, vaginal, by inhalation, via an implanted reservoir, or via a transdermal patch, and the like. Administration includes self-administration and the administration by another.

As used herein, the term “comprising” is intended to mean that the compositions and methods include the recited elements, but not excluding others. “Consisting essentially of” when used to define compositions and methods, shall mean excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions of this invention. Embodiments defined by each of these transition terms are within the scope of this invention.

A “control” is an alternative subject or sample used in an experiment for comparison purposes. A control can be “positive” or “negative.”

“Decrease” can refer to any change that results in a lower level of gene expression, protein expression, amount of a symptom, disease, composition, condition, or activity. A substance is also understood to decrease the level of the gene, the protein, the composition, or the amount of the condition when the level of the gene, the protein, the composition, or the amount of the condition is less/lower relative to the output of the level of the gene, the protein, the composition, or the amount of the condition without the substance. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom, Thus, a gene encodes a protein if transcription and translation of mRNA.

The “fragments,” whether attached to other sequences or not, can include insertions, deletions, substitutions, or other selected modifications of particular regions or specific amino acids residues, provided the activity of the fragment is not significantly altered or impaired compared to the nonmodified peptide or protein. These modifications can provide for some additional property, such as to remove or add amino acids capable of disulfide bonding, to increase its bio-longevity, to alter its secretory characteristics, etc.

The term “gene” or “gene sequence” refers to the coding sequence or control sequence, or fragments thereof. A gene may include any combination of coding sequence and control sequence, or fragments thereof. Thus, a “gene” as referred to herein may be all or part of a native gene. A polynucleotide sequence as referred to herein may be used interchangeably with the term “gene”, or may include any coding sequence, non-coding sequence or control sequence, fragments thereof, and combinations thereof.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or higher identity over a specified region when compared and aligned for maximum correspondence over a comparison window or designated region) as measured using a BLAST or BLAST 2.0 sequence comparison algorithms with default parameters described below, or by manual alignment and visual inspection (see, e.g., NCBI web site or the like). Such sequences are then said to be “substantially identical.” This definition also refers to, or may be applied to, the compliment of a test sequence. The definition also includes sequences that have deletions and/or additions, as well as those that have substitutions. As described below, the preferred algorithms can account for gaps and the like. Preferably, identity exists over a region that is at least about 10 amino acids or 20 nucleotides in length, or more preferably over a region that is 10-50 amino acids or 20-50 nucleotides in length. As used herein, percent (%) nucleotide sequence identity is defined as the percentage of amino acids in a candidate sequence that are identical to the nucleotides in a reference sequence, after aligning the sequences and introducing gaps, if necessary, to achieve the maximum percent sequence identity. Alignment for purposes of determining percent sequence identity can be achieved in various ways that are within the skill in the art, for instance, using publicly available computer software such as BLAST, BLAST-2, ALIGN, ALIGN-2 or Megalign (DNASTAR) software. Appropriate parameters for measuring alignment, including any algorithms needed to achieve maximal alignment over the full-length of the sequences being compared can be determined by known methods.

For sequence comparisons, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

One example of an algorithm that is suitable for determining percent sequence identity and sequence similarity are the BLAST and BLAST 2.0 algorithms, which are described in Altschul et al. (1977) Nuc. Acid Res. 25:3389-3402, and Altschul et al. (1990) J. Mol. Biol. 215:403-410, respectively. Software for performing BLAST analyses is publicly available through the National Center for Biotechnology Information (www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1990) J. Mol. Biol. 215:403-410). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) or 10, M=5, N=−4 and a comparison of both strands. For amino acid sequences, the BLASTP program uses as defaults a wordlength of 3, and expectation (E) of 10, and the BLOSUM62 scoring matrix (see Henikoff and Henikoff (1989) Proc. Natl. Acad. Sci. USA 89:10915) alignments (B) of 50, expectation (E) of 10, M=5, N=−4, and a comparison of both strands.

The BLAST algorithm also performs a statistical analysis of the similarity between two sequences (see, e.g., Karlin and Altschul (1993) Proc. Natl. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01.

“Inhibit”, “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100/o, or any amount of reduction in between as compared to native or control levels.

As used herein, the terms “may,” “optionally,” and “may optionally” are used interchangeably and are meant to include cases in which the condition occurs as well as cases in which the condition does not occur. Thus, for example, the statement that a formulation “may include an excipient” is meant to include cases in which the formulation includes an excipient as well as cases in which the formulation does not include an excipient.

“Operably linked”, as used herein, means at least two chemical structures joined together in such a way as to remain linked through the various manipulations described herein. Typically, the functional moiety and the encoding oligonucleotide are linked covalently via an appropriate linking group. The linking group is at least a bivalent moiety with a site of attachment for the oligonucleotide and a site of attachment for the functional moiety. For example, when the functional moiety is a polyamide compound, the polyamide compound can be attached to the linking group at its N-terminus, its C-terminus or via a functional group on one of the side chains. The linking group is sufficient to separate the polyamide compound and the oligonucleotide by at least one atom and in some embodiments by more than one atom. In some embodiments, the linking group is sufficiently flexible to allow the polyamide compound to bind target molecules in a manner which is independent of the oligonucleotide.

“Recombinant” used in reference to a gene refers herein to a sequence of nucleic acids that are not naturally occurring in the genome. The non-naturally occurring sequence may include a recombination, substitution, deletion, or addition of one or more bases with respect to the nucleic acid sequence originally present in the natural genome.

“Pharmaceutically acceptable” component can refer to a component that is not biologically or otherwise undesirable, i.e., the component may be incorporated into a pharmaceutical formulation of the invention and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic, and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents.

As used herein, the term “carrier” encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in Philadelphia, Lippincott, Williams & Wilkins, Philadelphia, Pa., 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, N.J.), polyethylene glycol (PEG), and PLURONICS™ (BASF; Florham Park, N.J.). To provide for the administration of such dosages for the desired therapeutic treatment, compositions disclosed herein can advantageously comprise between about 0.1% and 99% by weight of the total of one or more of the subject compounds based on the weight of the total composition including carrier or diluent.

The term “polynucleotide” refers to a single or double stranded polymer composed of nucleotide monomers.

The term “polypeptide” refers to a compound made up of a single chain of D- or L-amino acids or a mixture of D- and L-amino acids joined by peptide bonds.

The terms “peptide,” “protein,” and “polypeptide” are used interchangeably to refer to a natural or synthetic molecule comprising two or more amino acids linked by the carboxyl group of one amino acid to the alpha amino group of another.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition. The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition refers to an amount that is effective to achieve a desired therapeutic result. In some embodiments, a desired therapeutic result is the treatment of GVHD and/or a symptom thereof. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as coughing relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

The terms “treat,” “treating,” “treatment,” and grammatical variations thereof as used herein, include partially or completely delaying, alleviating, mitigating or reducing the intensity of one or more attendant symptoms of a disorder or condition and/or alleviating, mitigating or impeding one or more causes of a disorder or condition. Treatments according to the invention may be applied preventively, prophylactically, palliatively, or remedially. Prophylactic treatments are administered to a subject prior to onset (e.g., before obvious signs of GVHD), during early onset (e.g., upon initial signs and symptoms of GVHD), or after an established development of disease. Prophylactic administration can occur for several days to years prior to the manifestation of symptoms of an infection.

As used herein, the term “subject”, “patient”, or “host” can refer to living organisms such as mammals, including, but not limited to humans, livestock, dogs, cats, and other mammals. Administration of the therapeutic agents can be carried out at dosages and for periods of time effective for treatment of a subject. In some embodiments, the subject is a human.

### EGFL7 Peptides

In some aspects, disclosed herein is a recombinant endothelial growth factor like 7 (EGFL7) peptide or a fragment thereof. In some embodiments, the EGFL7 peptide comprises a polypeptide sequence at least 60% (e.g., at least 60%, at least 65%, at least 70%, at least 75%, at least 80%, at least 85%, at least 90%, at least 95%, at least 96%, at least 97%, at least 98%, at least 99%, or at least 99.5%) identical to SEQ ID NO: 1. In some embodiments, the EGFL7 peptide comprises the amino acid sequence of SEQ ID NO: 1.

In some embodiments, the EGFL7 peptide comprises a polypeptide sequence comprising at least 5 consecutive amino acids, at least 6 consecutive amino acids, at least 7 consecutive amino acids, at least 8 consecutive amino acids, at least 9 consecutive amino acids, at least 10 consecutive amino acids, at least 12 consecutive amino acids, at least 14 consecutive amino acids, at least 16 consecutive amino acids, at least 18 consecutive amino acids, at least 20 consecutive amino acids, at least 22 consecutive amino acids, at least 24 consecutive amino acids, at least 26 consecutive amino acids, at least 28 consecutive amino acids, at least 30 consecutive amino acids, at least 34 consecutive amino acids, at least 36 consecutive amino acids, at least 40 consecutive amino acids of SEQ ID NO: 1.

In some aspects, disclosed herein is a recombinant nucleic acid comprising a polypeptide encoding the EGFL7 peptide of any preceding aspect or a fragment thereof.

In some aspects, disclosed herein is a vector comprising a recombinant nucleic acid of any preceding aspect.

There are numerous variants of the EGFL7 peptide herein contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Substitutions, deletions, insertions or any combination thereof may be combined to arrive at a final construct.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations.

In some embodiments, the EGFL7 peptide of any preceding aspect is formulated in a pharmaceutically acceptable carrier. The tablets, troches, pills, capsules, and the like can also contain the following: binders such as gum tragacanth, acacia, corn starch or gelatin; diluents such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring can be added. When the unit dosage form is a capsule, it can contain, in addition to materials of the above type, a liquid carrier, such as a vegetable oil or a polyethylene glycol. Various other materials can be present as coatings or to otherwise modify the physical form of the solid unit dosage form. For instance, tablets, pills, or capsules can be coated with gelatin, wax, shellac, or sugar and the like. A syrup or elixir can contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound can be incorporated into sustained-release preparations and devices.

Compounds and compositions disclosed herein, including pharmaceutically acceptable salts or prodrugs thereof, can be administered intravenously, intramuscularly, or intraperitoneally by infusion or injection. Solutions of the active agent or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations can contain a preservative to prevent the growth of microorganisms.

The pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, for example, parabens, chlorobutanol, phenol, sorbic acid, thimerosal, and the like. In many cases, it will be preferable to include isotonic agents, for example, sugars, buffers sodium chloride. Prolonged absorption of the injectable compositions can be brought about by the inclusion of agents that delay absorption, for example, aluminum monostearate and gelatin.

Sterile injectable solutions are prepared by incorporating a compound and/or agent disclosed herein in the required amount in the appropriate solvent with various other ingredients enumerated above, as required, followed by filter sterilization. In the case of sterile powders for the preparation of sterile injectable solutions, the preferred methods of preparation are vacuu drying and the freeze drying techniques, which yield a powder of the active ingredient plus any additional desired ingredient present in the previously sterile-filtered solutions.

In some embodiments, the EGFL7 peptide of any preceding aspect is formulated in a nanoparticle. In general, a “nanoparticle” refers to any particle having a diameter of less than 1000 nm, e.g. about 10 nm to about 200 nm. Disclosed therapeutic nanoparticles may include nanoparticles having a diameter of about 60 to about 120 nm, or about 70 to about 130 nm, or about 60 to about 140 nm. Nanoparticles disclosed herein include one, two, three or more biocompatible and/or biodegradable polymers. For example, a contemplated nanoparticle may include about 10 to about 99 weight percent of a one or more block co-polymers that include a biodegradable polymer and polyethylene glycol, and about 0 to about 50 weight percent of a biodegradable homopolymer.

The term “polymer” as used herein refers to a relatively high molecular weight organic compound, natural or synthetic, whose structure can be represented by a repeated small unit, the monomer. Synthetic polymers are typically formed by addition or condensation polymerization of monomers. The polymers used or produced in the present invention are biodegradable. The polymer is suitable for use in the body of a subject, i.e. is biologically inert and physiologically acceptable, non-toxic, and is biodegradable in the environment of use, i.e. can be resorbed by the body. Examples of synthetic polymers include, but are not limited to, poly-lactic acid (PLA); polycaprolactone (PCL); polystyrene (PS); polyacrylamide; polyacrylate; poly (alkyl cyanoacrylates); poly (isobutyl cyanoacrylates); poly (butylcyanoacrylates); poly methyl (methcyanoacrylates); and combinations thereof.

### Methods of Use

In some aspects, disclosed herein is a method of treating or preventing graft-versus-host disease (GVHD) in a subject, comprising administering to the subject a therapeutically effective amount of a recombinant endothelial growth factor like 7 (EGFL7) peptide or fragment thereof. In some embodiments, the GVHD is acute GVHD.

In some aspects, disclosed herein is a method of treating an inflammatory disorder in a subject, comprising administering to the subject a therapeutically effective amount of a recombinant endothelial growth factor like 7 (EGFL7) peptide or fragment thereof. In some embodiments, the inflammatory disorder is inflammatory bowel disease. In some embodiments, the inflammatory disorder is Crohn's disease. In some embodiments, the inflammatory disorder is colitis. In some embodiments, the inflammatory disorder is an arthritic condition (e.g., rheumatoid arthritis, scleroderma, or lupus erythematosus), an endocrine condition (e.g., diabetes mellitus), a neurologic condition (e.g., multiple sclerosis or myasthenia gravis), a gastrointestinal condition (e.g., Crohn's disease or ulcerative colitis), or a hematological disorder (e.g., autoimmune hemolytic anemia).

In some embodiments, the EGFL7 peptide comprises a polypeptide sequence at least 60% (e.g., at least 60%, at least 65%, at least 70%, at least 75%, at least 80%, at least 85%, at least 90%, at least 95%, at least 96%, at least 97%, at least 98%, at least 99%, or at least 99.5%) identical to SEQ ID NO: 1. In some embodiments, the EGFL7 peptide comprises the amino acid sequence of SEQ ID NO: 1.

In some embodiments, the EGFL7 peptide comprises a polypeptide sequence comprising at least 5 consecutive amino acids, at least 6 consecutive amino acids, at least 7 consecutive amino acids, at least 8 consecutive amino acids, at least 9 consecutive amino acids, at least 10 consecutive amino acids, at least 12 consecutive amino acids, at least 14 consecutive amino acids, at least 16 consecutive amino acids, at least 18 consecutive amino acids, at least 20 consecutive amino acids, at least 22 consecutive amino acids, at least 24 consecutive amino acids, at least 26 consecutive amino acids, at least 28 consecutive amino acids, at least 30 consecutive amino acids, at least 34 consecutive amino acids, at least 36 consecutive amino acids, at least 40 consecutive amino acids of SEQ ID NO: 1. In some embodiments, the EGFL7 peptide fragment comprises a polypeptide sequence at least 60% (e.g., at least 60%, at least 65%, at least 70%, at least 75%, at least 80%, at least 85%, at least 90%, at least 95%, at least 96%, at least 97%, at least 98%, at least 99%, or at least 99.5%) identical to the corresponding fragment of SEQ ID NO: 1.

As used herein, the term “graft-versus-host” or “GVH” refers to an immune response of graft (donor) cells against host cells and tissues.

As used herein, the term “graft-vs-host disease” or “GVHD” refers to a condition, including acute and chronic, resulting from transplanted (graft) cell effects on host cells and tissues resulting from GVH. In other words, donor immune cells infused within the graft or donor immune cells that develop from the stem cells, may see the patient's (host) cells as foreign and turn against them with an immune response. As examples, patients who have had a blood or marrow transplant from someone else are at risk of having acute GVHD. Even donors who are HLA-matched with the recipient can cause GVHD because the donor cells can potentially also make an immune response against minor antigen differences in the recipient. Acute graft-versus-host disease (GVHD) is a disorder caused by donor immune cells in patients who have had an allogeneic marrow or blood cell transplantation. The most commonly affected tissues are skin intestine and liver. In severe cases, GVHD can cause blistering in the skin or excessive diarrhea and wasting. Also, inflammation caused by donor immune cells in the liver can cause obstruction that causes jaundice. Other tissues such as lung and thymus may also become affected. The diagnosis is usually confirmed by looking at a small piece of skin, liver, stomach or intestine with a microscope for observation of specific inflammatory characteristics. The symptoms of acute GVHD further comprises an increase of white blood cell counts and proinflammatory cytokine levels. The symptoms of acute GVHD usually begins within the first 3 months after the transplant. In some cases, it can persist, come back or begin more than 3 months after the transplant. For chronic GVHD, symptoms normally appear after 100 days post-transplant. In some cases, chronic GVHD appears much later, i.e., several months or years after allogeneic stem cell transplantation. Skin, mouth mucosa, eyes, gut, pain muscles and joints are typically symptoms of chronic GVHD. Chronic GVHD can lead to permanent damage to organs.

The compositions and methods disclosed herein for treating GVHD may be a treatment of one or more of blistering in the skin, skin rashes, abdominal cramps, excessive diarrhea, inflammation in the liver, intestine, lung, thymus, jaundice, and/or nausea.

Further, it should be understood herein that gut crypt cells are involved in self-renewal of the intestinal mucosa and usually found damaged in GVHD patients. The compositions and methods disclosed herein surprisingly increase the amount of gut crypt cells.

In some embodiments, the compositions and methods disclosed herein induces T cell exhaustion and/or T cell tolerance in the subject. “T cell exhaustion” refers to the loss or the reduction of effector function and/proliferation capacity of T lymphocytes. Exhausted T cells are typically identified by the increased expression of markers including, for example, PD-1 (UniProtKB: Q15116), TIM-3 (UniProtKB: Q8TDQO), 2B4 (UniProtKB: Q9BZW8), and LAG3 (UniProtKB: P18627), and the less production of cytokines including, for example, IL-2 (UniProtKB: P60568), TNF-α (UniProtKB: P01375), IFN-γ (UniProtKB: P01579), and Granzyme B (UniProtKB: P10144). The compositions and methods disclosed herein result in an increased number of exhausted T cells.

“T cell tolerance” refers to a state of unresponsiveness in which the lymphocytes remain alive but cannot exert effector functions against a particular antigen. Tolerated T cell or regulatory T cell are typically identified by the increased expression of CD25 (UniProtKB: P01589) and transcriptional factor FOXP3 (UniProtKB: Q9BZS1), and the increased production of cytokines including, for example, IL-10 (UniProtKB: P22301) and TGF-β (UniProtKB: P01137). The compositions and methods disclosed herein results in an increased number of regulatory T cells.

Accordingly, it should be understood herein that the compositions and methods disclosed herein decreases the levels of one or more inflammatory cytokines. In some embodiments, the one or more inflammatory cytokines comprise TNF-α or IFN-γ. In some embodiments, the compositions and methods disclosed herein decreases levels of ICAM-1 (UniProtKB: P05362) or VCAM-1 (UniProtKB: P19320). In some embodiments, the recombinant EGFL7 peptide is formulated in a pharmaceutically acceptable carrier.

Administration can be carried out by any suitable route, including oral, topical, intravenous, subcutaneous, transcutaneous, transdermal, intramuscular, intra-joint, parenteral, intra-arteriole, intradermal, intraventricular, intracranial, intraperitoneal, intralesional, intranasal, rectal, intravaginal, by inhalation, via an implanted reservoir, or via a transdermal patch, and the like.

It should be understood the disclosed methods of treating, preventing, reducing, and/or inhibiting GVHD and/or a symptom thereof comprising administering to a subject a therapeutically effective amount of the recombinant EGFL7 peptide disclosed herein or a fragment thereof, can be used prior to or following the onset of GVHD and/or a symptom thereof, to treat, prevent, inhibit, and/or reduce GVHD In some embodiments, the recombinant EGFL7 peptide disclosed herein or a fragment thereof can be used prior to bone marrow transplantation, following bone marrow transplantation and prior to the onset of GVHD and/or any symptom thereof. In one aspect, the disclosed methods can be employed 12, 11, 10, 9, 8, 7, 6, 5, 4, 3, 2, 1 months, 30, 29, 28, 27, 26, 25, 24, 23, 22, 21, 20, 19, 18, 17, 16, 15, 14, 13, 12, 11, 10, 9, 8, 7, 6, 5, 4, 3 days, 60, 48, 36, 30, 24, 18, 15, 12, 10, 9, 8, 7, 6, 5, 4, 3, 2 hours, 60, 45, 30, 15, 10, 9, 8, 7, 6, 5, 4, 3, 2, or 1 minute prior to bone marrow transplantation. In some aspects, the disclosed methods can be employed 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days, 4, 5, 6, 7, 8, 9, 10, 11, 12 or more months, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after bone marrow transplantation. In some aspects, the disclosed methods can be employed 1, 2, 3, 4, 5, 6, 7, 8, 9, 10, 15, 20, 25, 30, 35, 40, 45, 50, 55, 60, 75, 90, 105, 120 minutes, 3, 4, 5, 6, 7, 8, 9, 10, 11, 12, 15, 18, 24, 30, 36, 48, 60 hours, 3, 4, 5, 6, 7, 8, 9, 10, 11, 12, 13, 14, 15, 16, 17, 18, 19, 20, 21, 22, 23, 24, 25, 26, 27, 28, 29, 30, 45, 60, 90 or more days, 4, 5, 6, 7, 8, 9, 10, 11, 12 or more months, 30, 29, 28, 27, 26, 25, 24, 23, 22, 21, 20, 19, 18, 17, 16, 15, 14, 13, 12, 11, 10, 9, 8, 7, 6, 5, 4, 3, 2, 1 years after onset of GVHD and/or a symptom thereof.

In some embodiments, the method of any preceding aspect further comprises administering to the subject a therapeutically effective amount of an additional agent for treating GVHD. In some embodiments, the additional agent is selected from the group consisting of methotrexate, cyclosporine, tacrolimus, mycophenolate mofetil, sirolimus, corticosteroid, antithymocyte globulin, alemtuzumab, and cyclophosphamide. In some embodiments, the additional agent is selected from the group consisting of azathioprine, pentostatin (deoxycoformycin, Nipent), infliximab, dacluzimab, and ibrutinib (Imbruvica).

In some embodiments, the GVHD to be treated is refractory chronic GVHD.

In some aspects, disclosed herein is a method for prevention or treatment of acute graft-versus-host-disease in a patient that has received or is about to receive an allogeneic hematopoietic stem cell transplant, the method comprising administering to the subject a therapeutically effective amount of a recombinant endothelial growth factor like 7 (EGFL7) peptide or fragment thereof.

In some embodiments, the administration of the recombinant endothelial growth factor like 7 (EGFL7) peptide or fragment thereof to the subject is initiated on the day of allogeneic hematopoietic stem cell transplantation

In some embodiments, the administration of the recombinant endothelial growth factor like 7 (EGFL7) peptide or fragment thereof to the subject is initiated when symptoms of GVDH appear after allogeneic hematopoietic stem cell transplantation.

In some aspects, disclosed herein is a method for prevention or treatment of graft-versus-host-disease or transplant rejection in a subject in need thereof, comprising administering to the subject a therapeutically effective amount of a recombinant endothelial growth factor like 7 (EGFL7) peptide or fragment thereof.

### EXAMPLES

The following examples are set forth below to illustrate the compositions, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1. Modulating Endothelial Cells with EGFL7 to Diminish aGVHD after Allogeneic Bone Marrow Transplantation in Mice

Acute graft versus host (aGVHD) is a leading cause of death after allogeneic-hematopoietic stem cell transplant (allo-HSCT), underscoring the need for novel therapies. Here, it was shown that treatment with recombinant EGFL7 (rEGFL7) in two different murine models of aGVHD decreases aGVHD severity and improves survival in recipient mice after allogeneic transplantation with respect to controls without affecting graft versus leukemia effect. Furthermore, it was shown that rEGFL7 treatment results in higher thymocytes, T, B and dendritic cells in recipient mice after allo-HSCT. This example shows the ability of rEGFL7 therapy to reduce GHVD severity and mortality after allo-HSCT.

### BACKGROUND

Allogeneic hematopoietic stem cell transplantation (allo-HSCT) is used to treat patients with high risk/refractory hematological malignancies and/or blood disorders. Unfortunately, acute graft-versus-host disease (aGVHD) is the principal complication of allo-HSCT. aGVHD is mediated by alloreactive donor T lymphocytes and occurs in response to differences in major and/or minor histocompatibility antigens expressed by recipient cells. Despite the use of standard GVHD prophylaxis regimens such as calcineurin inhibitors and other agents, 30-75% of allo-HSCT patients eventually develop aGVHD. Patients with severe and/or steroid-refractory aGVHD have a poor long-term prognosis and a mortality rate reaching 70-80%. Thus, alternative treatments for controlling excessive aGVHD are needed.

Epidermal growth factor-like domain 7 (EGFL7) is a ˜30KDa secreted protein important for angiogenesis and neurogenesis. EGFL7 inhibits endothelial cell (EC) activation by proinflammatory cytokines through a negative feedback loop. In a murine model of multiple sclerosis, EGFL7 reduced neuroinflammation by binding αvβ3 integrins and preventing T cell infiltration. Thus, unlike immunosuppressive therapies that target immune cell function, EGFL7 acts primarily on blood vessels to reduce immune cell infiltration. Given the importance of lymphocyte homing in aGVHD pathology, it was investigated in this example whether blocking endothelial cell (EC) activation after allo-HSCT can reduce GVHD severity. This study herein is the first to demonstrate EGFL7 as a novel treatment for allo-HSCT patients that develop aGVHD.

### Materials and Methods

Mice and reagents. C57BL/6.SJL (cat. #: 002014) and B6D2F1 (cat. #100006), BALB/c (cat. #000651), and C57Bl/6J (cat. #000664) mice were purchased from The Jackson Laboratory (Bar Harbor, Me.). All animals were housed at Maisonneuve-Rosemont Hospital and Ohio State University animal facilities. Animal studies were performed in accordance with the Maisonneuve-Rosemont Hospital Animal Care Committee and IACUC (Ohio State University). Recombinant human EGFL7 (rEGFL7) was purchased from Peprotech (Rockyhill, N.J., USA).

Bone marrow transplantation and GVHD. B6D2F1 mice were irradiated (2×600 rad, 3 hours apart) and 10⁷ BM cells from C57BL/6 donor mice (H-2^{b}) were injected intravenously into lethally irradiated B6D2F1 recipients (H-2^{bid}) along with 3×10⁶ T-cells from B6.SJL (allogeneic) or B6D2F1 (syngeneic) mice. T-cells were isolated using the T cell enrichment kit (StemCell Technologies). Mice were monitored daily and scored thrice a week for clinical severity of acute GVHD using a scoring system modified from Cooke et al. Vehicle (PBS) or rEGFL7 (10 μg) was administered intraperitoneally (I.P.) daily to mice, starting when signs of GVHD appear (˜17-21 days post-SCT). Mice with a clinical score of 7 or more were considered very sick and euthanized according to approved animal protocols.

Bone marrow transplantation and GVHD: For the C57BL/6 into BALB/c allo-SCT model, BALB/c recipient mice were irradiated (2×350 rad, 3 hours apart) on day −1. On the day of transplant, lethally irradiated BALB/c recipients received T cell depleted bone marrow cells (TCD-BM, 10×10⁶ cells) along with purified B6 T cells (1×10⁶). T cell depletion from BM cells was carried out by CD90 magnetic bead separation (Miltenyi Biotec). T cells were isolated from donor C57BL/6 spleen by negative selection using the Pan T cell isolation kit (Miltenyi Biotec). Two weeks after transplant, recipients were divided into 3 cohorts—no treatment, PBS vehicle or rEGFL7 administered intraperitoneally (IP) 5 times a week for 5 weeks.

Histopathological scoring and microscopy: Liver and intestine were harvested from euthanized mice, paraffin embedded and stained with hematoxylin & eosin (H&E). Stained sections were labeled without reference to treatment cohort and scored by a pathologist in a blinded fashion. A scoring method adapted and modified from Cooke et al.¹ was used to assess histological changes associated with aGVHD. For large bowel, the scoring method assessed six histological criteria such as villous blunting, crypt loss, crypt epithelial cell apoptosis, crypt regeneration, lamina propria inflammation, and mucosal ulceration. Each of the histological parameters of large bowel, aGVHD was scored on a point scale from 0 to 4 depending on the extent and severity of tissue injury (0—Normal or no change, 0.5—focal & rare, 1—focal & mild, 2—diffusely present but mild in intensity, 3—diffuse & moderate in intensity and 4—diffuse and severe in intensity). Similarly, the histological intensity of aGVHD in different liver sections was assessed on a similar point scale by evaluating seven parameters comprising portal tract inflammation, bile duct injury, vascular endotheliitis, periportal, or limiting plate necrosis, lobular necro-inflammatory activity, zonal necrosis, and sinusoidal lymphocytosis. The total score for the histological intensity of aGVHD in each organ was obtained by adding the scores for each of the parameters in the respective organ. Large intestine and liver paraffin sections were stained with anti-CD3 (Santa Cruz, SC-1127), anti-CD4 (Abcam, ab183685), anti-CD8 (Thermo Scientific, 14-0808-82), anti-CD31 (R&D, AF3628), and anti-Ki-67 (Thermo Scientific, 14-5698-82) antibodies and secondary antibodies conjugated with Alexa Fluor 488, Alexa 594, and Alexa Fluor 647 (Invitrogen) for immunofluorescence. Nuclei were counterstained with DAPI. Images were obtained using the Olympus FV3000 confocal microscope. Images were analyzed using NIH ImageJ software.

Flow cytometry. Cells were resuspended at a density of 10⁷ cells/ml in FACS buffer, incubated 30 min on ice with diluted monoclonal antibodies and then washed and resuspended in FACS buffer for immediate analysis. Monoclonal antibodies: APC-anti-CD4 (GK1.5); PeCy7-anti-CD8 (53-2.7); PerCPCy5.5-anti-CD45.1 (A20); PE- and FITC-anti-CD11c (N418); PerCPCy5.5-anti-CD11b (Ml/70); FITC-anti-TCRβ (H57-597); APC-anti-H2-K^{d} (SF1-1.1); APC-Cy7-anti IAIE (M5/114.15.2); PE-anti H2-K^{b} (AF6-88.5) were purchased from BioLegend: Cells were analyzed on a Fortessa (BD Biosciences) using FACSDiva software (BD Biosciences) or FlowJo software (TreeStar).

GVL experiments. B6D2F1 recipients were lethally irradiated (1200 cGy) in two doses (2×600cGy) to minimize toxicity on day −1. Firefly luciferase transduced P815 mastocytoma^{2,3} cells (5000 cells) were injected intravenously into F1 recipients on day 0 along with TCD-BM (10×10⁶ cells). B6 donor splenocytes (15×10⁶ cells) were administered intravenously on day +1 to treatment groups. Treatment groups included: PBS vehicle and rEGFL7 were administered daily starting day +3 until day +10 post-transplant. TCD-BM+P815 cells (leukemia alone) served as the control group. P815-induced leukemic death was defined by the occurrence of either macroscopic tumor nodules in liver and/or spleen or hind-leg paralysis. GVHD death was defined by the absence of leukemia and the presence of clinical and histopathological signs of GVHD.

In vivo imaging Xenogen IVIS imaging system (Caliper Life Sciences) was used for live animal imaging. Mice were anesthetized using 1.5% isofluorane (Piramal Healthcare). XenoLight RediJect D-Luciferin Ultra Bioluminescent Substrate (150 mg/kg body weight; 30 mg/mL in PBS; Perkin Elmer) was injected IP and IVIS imaging was performed 7-10 minutes after substrate injection. Whole body bioluminescent signal intensity was determined using IVIS Living Image software v4.3.1 (Caliper Life Sciences), and pseudocolor images overlaid on conventional photographs are shown. Data were analyzed and presented as photon counts per area.

Statistical analysis. Prism 5.0 (GraphPad Software) was used for all statistical analyses. Survival curves were analyzed using Kaplan-Meier and log-rank test methods. For estimating statistical significance in clinical scores using multiple t tests over time, P values were adjusted using the two-stage linear step-up procedure of Benjamini, Krieger, and Yekutieli⁴. Mann Whitney was used to compare pairs of data, and Kruskal-Wallis with Dunn's post-test was used to compare 3 or more groups. A p-value less than or equal to 0.05 was considered significant. Data is presented as mean±s.e.m.

### Results and Discussion

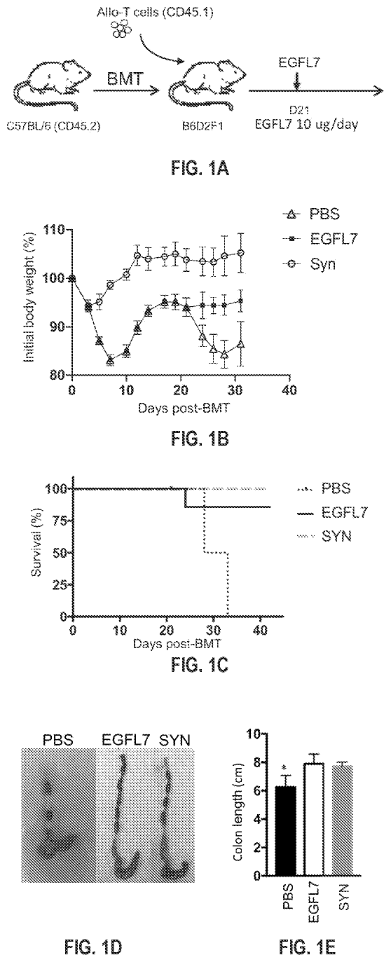

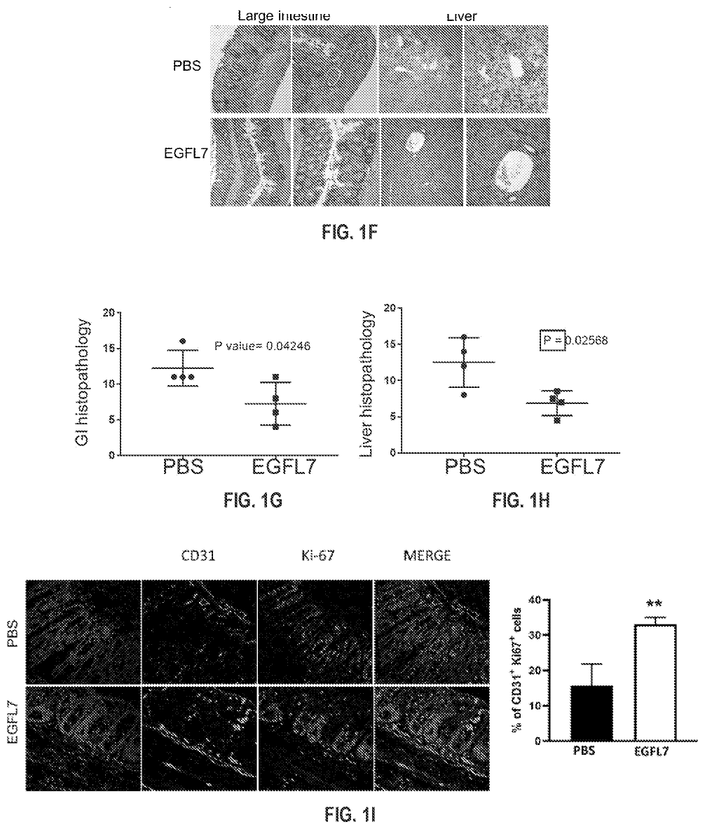

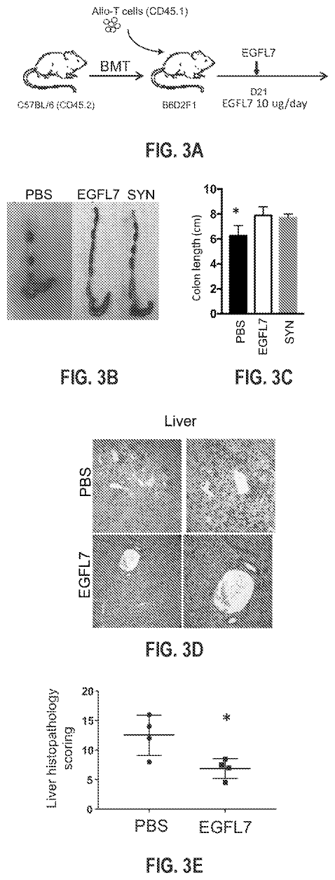

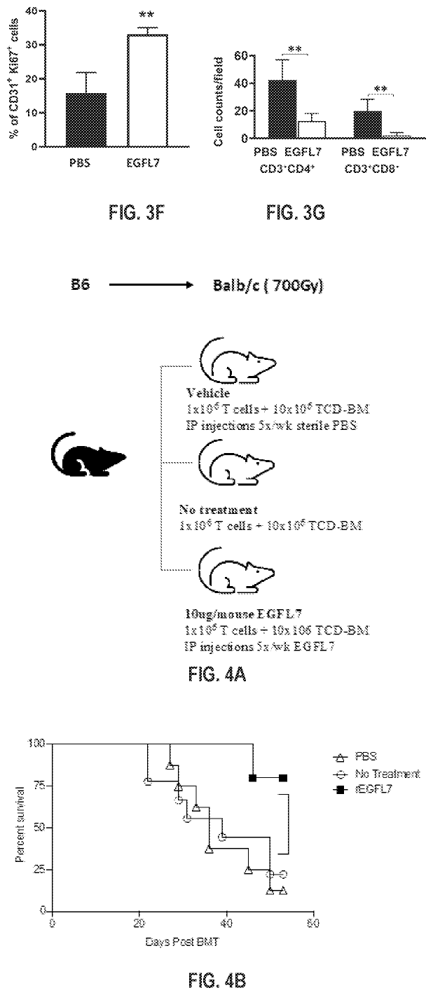

Current therapies for aGVHD rely largely on the inhibition of immune cell activation and effector functions. Here, EGFL7, an inhibitor of EC activation, was evaluated for reducing aGVHD. A parent into F1 (B6->B6D2F1) mouse model was used to evaluate the therapeutic benefit of EGFL7 treatment (FIG. 1A). Mice at day +21 post-HSCT showed evidence of mild hunching posture and dull fur. After 7 days of rEGFL7 treatment, mice showed marked improvement in posture, fur appearance and activity. This was accompanied by a stop or reduction in body weight-loss in rEGFL7-treated mice in contrast to PBS-treated mice that continued to lose weight (FIG. 1B). Importantly, rEGFL7 treatment prolonged survival of mice after allo-HSCT compared to PBS controls (FIG. 1C). This result was validated in a second mouse model of aGVHD, B6->Balb/c, showing increased survival of rEGFL7-treated mice compared to PBS controls (FIG. 3). The gut and liver are typically affected by aGVHD18,19. Visual examination of the colon revealed the presence of swelling and a reduction in the length in PBS group compared with rEGFL7-treated mice (FIG. 1D-E). Histopathology analysis revealed higher amount of leukocyte infiltration in both liver and large intestine of PBS group compared to rEGFL7-treated mice (FIG. 1F-G). Large intestine of PBS-treated mice showed focal to diffuse areas of villous atrophy with a dense amount of mixed inflammatory infiltrate in the lamina propria composed predominantly of lymphomononuclear cells and neutrophils. In addition, PBS-treated mice showed a more profound loss of mucous secreting goblet cells in the mucosal epithelium along with enhanced crypt epithelial cell apoptosis (>1 apoptotic body/crypt), and focal crypt abscess formation. In contrast, rEGFL7-treated mice showed normal integrity of mucosal epithelium with negligible to mild degree of lamina propria inflammation and a lesser number of crypt epithelial cell apoptotic bodies (FIG. 1F). In the liver, rEGFL7-treated mice showed less number and limited extent of portal tracts involvement by inflammatory cell infiltrates, less degree of bile duct injury, lobular necro-inflammatory activity, and vascular endothelitis (FIG. 1F). Thus, reduction in leukocyte infiltration in the gut and liver is consistent with an overall lower aGVHD histopathological score in rEGFL7-treated mice compared to PBS-treated group (FIG. 1G-H). Finally, immunofluorescence microscopy was performed to assess EC and T lymphocyte populations in the intestine. It was found that rEGFL7 treatment resulted in increased proliferation (Ki-67+) of intestine ECs (CD31+) (FIG. 1I) with an associated decrease in infiltrating CD4+ and CD8+ T-cells (Figure J). ECs damage occurs during GVHD and based on these data, it was shown that rEGFL7 treatment not only reduces EC activation but perhaps promotes angiogenesis/repair, leading to reduction in T cell infiltration in the gut and liver, resulting in GVHD improvement.

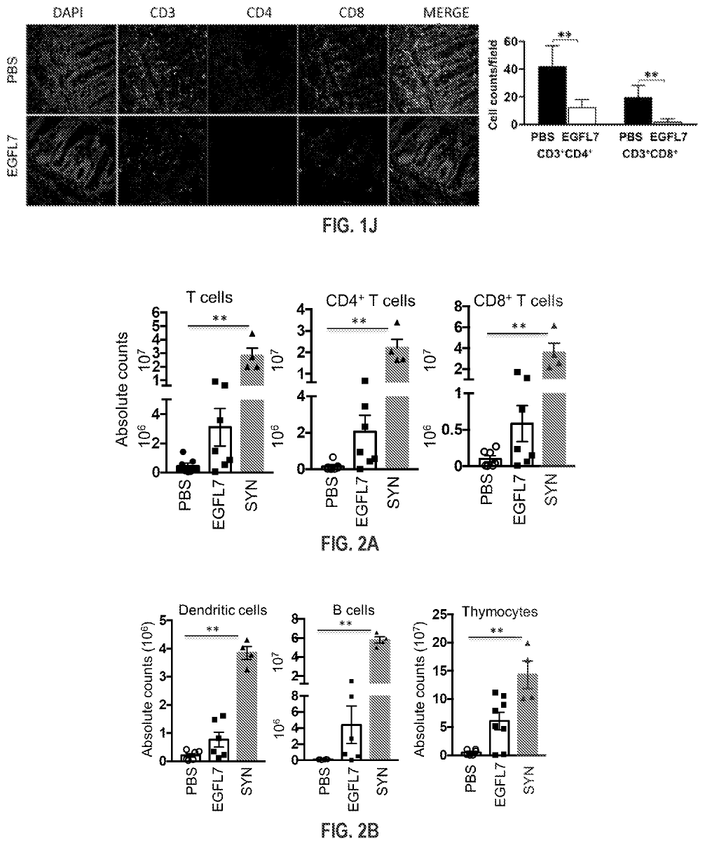

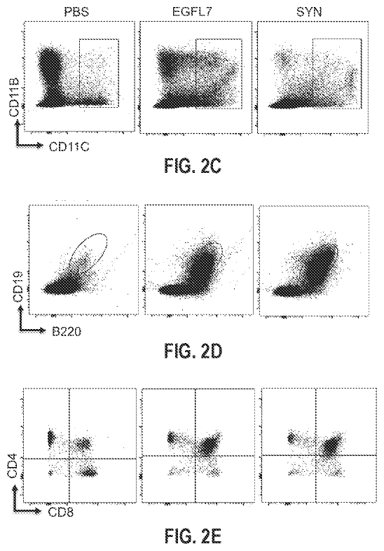

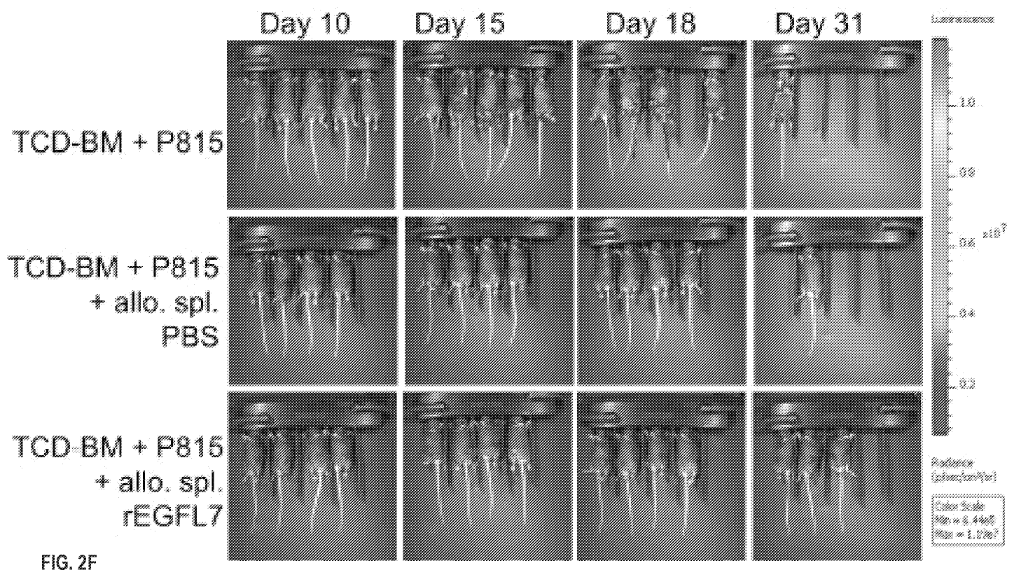

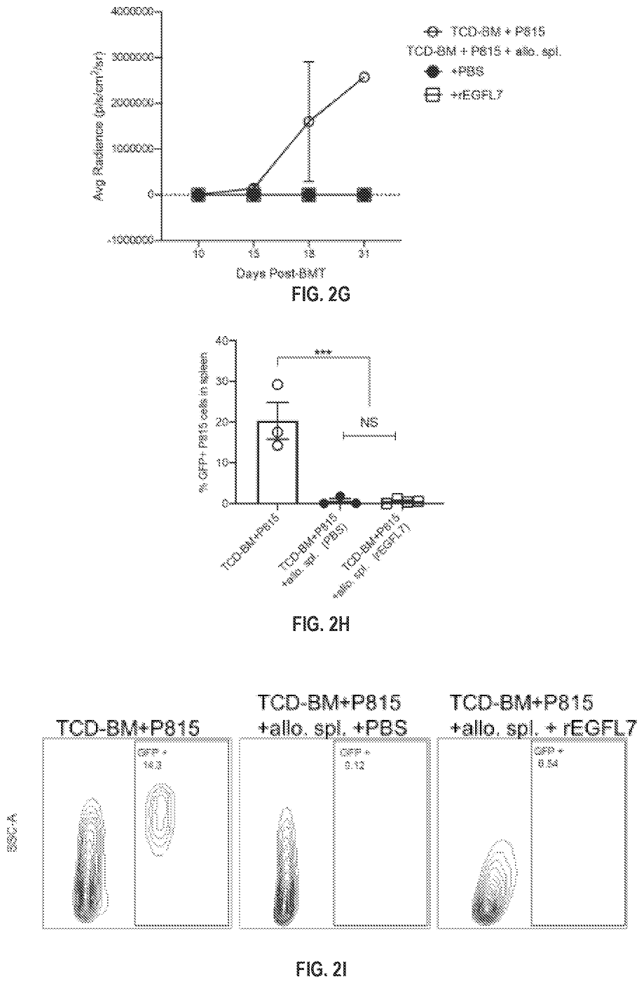

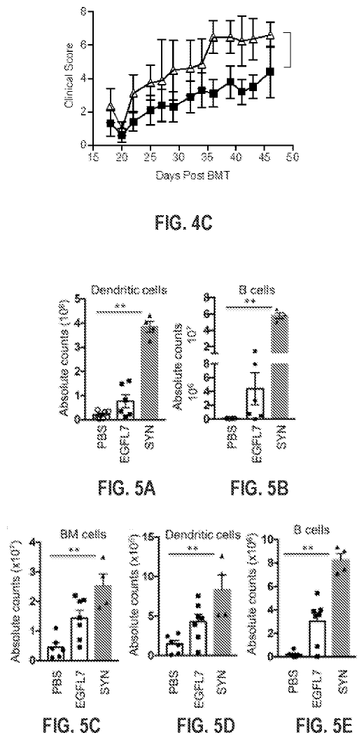

aGVHD insults to primary and secondary lymphoid organs impairs immune reconstitution after allo-HSCT. Treatment with rEGFL7 tends to increase T cell (FIG. 2A), B cell and dendritic cell (DC) counts in the spleen (FIG. 2B-D) and BM (FIG. 4) compared with PBS-treated mice. rEGFL7 therapy also tends to increase thymocyte counts with normalization of double-positive and single-positive CD4+ and CD8+ thymocyte populations in rEGFL7 compared with PBS-treated mice (FIGS. 2B and 2E). Variability in immune reconstitution may be related to GVHD insults prior to initiating EGFL7 treatment. Additional studies are needed. Thus, rise in T, B and DC counts is consistent with a reduction in aGVHD severity in rEGFL7-treated mice. Part of the success of an allogeneic transplant is the ability to sustain the graft-versus-leukemia (GVL) effect important for eliminating minimal residual disease. To study the impact of rEGFL7 treatment on GVL, a luciferase-transduced murine mastocytoma P815 cell line was transplanted along with or without alloreactive-B6 splenocytes. Recipients of allogeneic splenocytes were treated daily with PBS or rEGFL7 starting at day +3 until day +10 post-BMT. Mice transplanted with mastocytoma P815 cells without allogeneic splenocytes all succumbed to leukemia. In mice that received allogeneic splenocytes, treatment with rEGFL7 did not interfere with GVL effects as seen by decreased leukemia burden similar to PBS controls (FIG. 2F, G). Using flow cytometry, the absence of P815 cells (GFP+) in the spleen of rEGFL7-treated mice was confirmed (FIG. 2H, I) further showing the GVL-sparing effect of rEGFL7.

In conclusion, EGFL7 was shown to reduce/treat aGVHD. While immunosuppressive therapies typically impair immune cell activation/functions, the effectiveness of EGFL7 therapy to treat GVHD relies largely on the inhibition of EC activation and access of immune cells to inflamed tissues. This work represents the first determination that EGFL7 therapy can diminish/treat aGVHD and provides a novel perspective about targeting EC activation to reduce GVHD.

### REFERENCES

1. Ferrara J L, Deeg H J. Graft-versus-host disease. N Engl J Med. 1991; 324(10):667-674.
2. Teshima T, Ordemann R, Reddy P, et al. Acute graft-versus-host disease does not require alloantigen expression on host epithelium. Nat Med. 2002; 8(6):575-581.
3. Matte C C, Liu J, Cormier J, et al. Donor APCs are required for maximal GVHD but not for GVL. Nat Med. 2004; 10(9):987-992.
4. Hill L, Alousi A, Kebriaei P, Mehta R, Rezvani K, Shpall E. New and emerging therapies for acute and chronic graft versus host disease. Ther Adv Hematol. 2018; 9(1):21-46.
5. Holtan S G, Pasquini M, Weisdorf D J. Acute graft-versus-host disease: a bench-to-bedside update. Blood. 2014; 124(3):363-373.
6. Castilla-Llorente C, Martin P J, McDonald G B, et al. Prognostic factors and outcomes of severe gastrointestinal GVHD after allogeneic hematopoietic cell transplantation. Bone Marrow Transplant. 2014:49(7):966-971.
7. Martin P J, Rizzo J D, Wingard J R, et al. First- and second-line systemic treatment of acute graft-versus-host disease: recommendations of the American Society of Blood and Marrow Transplantation. Biol Blood Marrow Transplant. 2012; 18(8):1150-1163.
8. Pidala J, Kim J, Roman-Diaz J, et al. Pentostatin as rescue therapy for glucocorticoid-refractory acute and chronic graft-versus-host disease. Ann Transplant. 2010; 15(4):21-29.
9. Oh J, Park S H, Lee T S, Oh H K, Choi J H, Choi Y S. High expression of epidermal growth factor-like domain 7 is correlated with poor differentiation and poor prognosis in patients with epithelial ovarian cancer. J Gynecol Oncol. 2014; 25(4):334-341.
10. Bambino K, Lacko L A, Hajjar K A, Stuhlmann H. Epidermal growth factor-like domain 7 is a marker of the endothelial lineage and active angiogenesis. Genesis. 2014; 52(7):657-670.
11. Nichol D, Stuhlmann H. EGFL7: a unique angiogenic signaling factor in vascular development and disease. Blood. 2012; 119(6):1345-1352.
12. Pinte S, Caetano B, Le Bras A, et al. Endothelial Cell Activation Is Regulated by Epidermal Growth Factor-like Domain 7 (Egfl7) during Inflammation. J Biol Chem. 2016; 291(46):24017-24028.
13. Larochelle C, Uphaus T, Broux B, et al. EGFL7 reduces CNS inflammation in mouse. Nat Commun. 2018; 9(1):819.
14. Sackstein R. A revision of Billingham's tenets: the central role of lymphocyte migration in acute graft-versus-host disease. Biol Blood Marrow Transplant. 2006; 12 (1 Suppl 1):2-8.
15. Nomura S, Ishii K, Fujita S, Nakaya A, Satake A, Ito T. Associations between acute GVHD-related biomarkers and endothelial cell activation after allogeneic hematopoietic stem cell transplantation. Transpl Immunol. 2017; 43-44:27-32.
16. Cooke K R, Kobzik L, Martin T R, et al. An experimental model of idiopathic pneumonia syndrome after bone marrow transplantation: 1. The roles of minor H antigens and endotoxin. Blood. 1996:88(8):3230-3239.
17. Coutinho A E, Chapman K E. The anti-inflammatory and immunosuppressive effects of glucocorticoids, recent developments and mechanistic insights. Mol Cell Endocrinol. 2011; 335(1):2-13.
18. Hill G R, Ferrara J L. The primacy of the gastrointestinal tract as a target organ of acute graft-versus-host disease. rationale for the use of cytokine shields in allogeneic bone marrow transplantation. Blood. 2000; 95(9):2754-2759.
19. Shulman H M, Sharma P, Amos D, Fenster L F, McDonald G B. A coded histologic study of hepatic graft-versus-host disease after human bone marrow transplantation. Hepatology. 1988:8(3):463-470.
20. Biedermann B C, Sahner S, Gregor M, et al. Endothelial injury mediated by cytotoxic T lymphocytes and loss of microvessels in chronic graft versus host disease. Lancet. 2002; 359(9323):2078-2083.
21. Mir E, Palomo M, Rovira M, et al. Endothelial damage is aggravated in acute GvHD and could predict its development. Bone Marrow Transplant. 2017; 52(9):1317-1325.
22. Mackall C L, Fleisher T A, Brown M R, et al. Age, thymopoiesis, and CD4+T-lymphocyte regeneration after intensive chemotherapy. N Engl J Med. 1995; 332(3):143-149.
23. Takebayashi M, Amakawa R, Tajima K, et al. Blood dendritic cells are decreased in acute graft-versus-host disease. Bone Marrow Transplant. 2004:33(10):989-996.
24. Shono Y, Ueha S, Wang Y, et al. Bone marrow graft-versus-host disease: early destruction of hematopoietic niche after MHC-mismatched hematopoietic stem cell transplantation. Blood. 2010; 115(26):5401-5411.
25. Gauthier S D, Leboeuf D, Manuguerra-Gagne R, Gaboury L, Guimond M. Stromal-Derived Factor-1alpha and Interleukin-7 Treatment Improves Homeostatic Proliferation of Naive CD4(+) T Cells after Allogeneic Stem Cell Transplantation. Biol Blood Marrow Transplant. 2015; 21(10):1721-1731.
26. Cooke K R, Kobzik L, Martin T R, et al. An experimental model of idiopathic pneumonia syndrome after bone marrow transplantation: I. The roles of minor H antigens and endotoxin. Blood. 1996; 88(8):3230-3239.
27. Teshima T, Hill G R, Pan L, et al. IL-11 separates graft-versus-leukemia effects from graft-versus-host disease after bone marrow transplantation. J Clin Invest. 1999; 104(3):317-325.
28. Tsukada N, Kobata T, Aizawa Y, Yagita H, Okumura K. Graft-versus-leukemia effect and graft-versus-host disease can be differentiated by cytotoxic mechanisms in a murine model of allogeneic bone marrow transplantation. Blood. 1999; 93(8):2738-2747.
29. Benjamini Y, Krieger A M, Yekutieli D. Adaptive linear step-up procedures that control the false discovery rate. Biometrika. 2006; 93(3):491-507.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

### SEQUENCES

SEQ ID NO: 1
MRGSQEVLLMWLLVLAVGGTEHAYRPGRRVCAVRAHGDPVSESFVQRVY
QPFLTTCDGHRACSTYRTIYRTAYRRSPGLAPARPRYACCPGWKRTSGL
PGACGAAICQPPCRNGGSCVQPGRCRCPAGWRGDTCQSDVDECSARRGG
CPQRCVNTAGSYWCQCWEGHSLSADGTLCVPKGGPPRVAPNPTGVDSAM
KEEVQRLQSRVDLLEEKLQLVLAPLHSLASQALEHGLPDPGSLLVHSFQ
QLGRIDSLSEQISFLEEQLGSCSCKKDS

1. A method of treating graft-versus-host disease (GVHD) in a subject, comprising administering to the subject a therapeutically effective amount of a recombinant endothelial growth factor like 7 (EGFL7) peptide or fragment thereof.

2. The method of claim 1, wherein the recombinant EGFL7 peptide comprises a polypeptide sequence at least 80% identical to SEQ ID NO: 1.

3. The method of claim 1, wherein the recombinant EGFL7 peptide comprises the polypeptide sequence of SEQ ID NO: 1.

4. The method of claim 1, wherein the recombinant EGFL7 peptide comprises a polypeptide sequence comprising at least 10 consecutive amino acids of SEQ ID NO: 1.

5. The method of claim 1, wherein the recombinant EGFL7 peptide is administered daily.

6. The method of claim 1, wherein the recombinant EGFL7 peptide is administered prior to and/or after bone marrow transplantation.

7. The method of claim 1, wherein the recombinant EGFL7 peptide is administered intravenously.

8. The method of claim 1, wherein the recombinant EGFL7 mitigates a symptom of GVHD, wherein the symptom comprises skin rashes, abdominal cramps, nausea, or liver injury.

9. The method of claim 1, wherein the recombinant EGFL7 peptide induces T cell exhaustion and/or T cell tolerance in the subject.

10. The method of claim 1, wherein the recombinant EGFL7 peptide decreases a level of an inflammatory cytokine and/or an adhesion molecule in the subject.

11. The method of claim 10, wherein the inflammatory cytokine comprises TNF-α or IFN-γ.

12. The method of claim 10, wherein the adhesion molecule comprises ICAM-1 or VCAM-1.

13. The method of claim 1, wherein the recombinant EGFL7 is formulated in a pharmaceutically acceptable carrier.

14. The method of claim 1, further comprising administering to the subject a therapeutically effective amount of an additional agent for treating GVHD.

15. The method of claim 14, wherein the additional agent is selected from the group consisting of methotrexate, cyclosporine, tacrolimus, mycophenolate mofetil, sirolimus, corticosteroid, antithymocyte globulin, alemtuzumab, and cyclophosphamide.


SEQUENCE LISTING (1 sequence)

SEQ ID NO: 1 - protein, 273 aa, Artificial Sequence
  Synthetic Construct
MRGSQEVLLMWLLVLAVGGTEHAYRPGRRVCAVRAHGDPVSESFVQRVYQPFLTTCDGHRACSTYRTIYRTAYRRSPGLAPARPRYACCPGWKRTSGLPGACGAAICQPPCRNGGSCVQPGRCRCPAGWRGDTCQSDVDECSARRGGCPQRCVNTAGSYWCQCWEGHSLSADGTLCVPKGGPPRVAPNPTGVDSAMKEEVQRLQSRVDLLEEKLQLVLAPLHSLASQALEHGLPDPGSLLVHSFQQLGRIDSLSEQISFLEEQLGSCSCKKDS